(19) United States
(12) Patent Application Publication
(10) Pub. No.: US 2025/0025875 A1
(43) Pub. Date: Jan. 23, 2025

(54) Fluid Pumping and Reversible Flow in Strain Driven Microfluidic Applications

(71) Applicant: Santa Clara University, Santa Clara, CA (US)

(72) Inventors: Ismail Emre Araci, San Jose, CA (US); Rana Altay, Santa Clara, CA (US)

(21) Appl. No.: 18/777,431

(22) Filed: Jul. 18, 2024

Related U.S. Application Data
(60) Provisional application No. 63/527,791, filed on Jul. 19, 2023.

(51) Int. Cl. B01L 3/00; A61M 35/00

(52) U.S. Cl. CPC B01L 3/50273; A61M 35/10; B01L 2200/027; B01L 2200/0689; B01L 2200/16; B01L 2400/0487

(57) ABSTRACT

A microfluidic device is provided that has a negative volumetric strain under a tensile strain. The microfluidic device is capable of pumping fluids stored inside a reservoir towards an outlet through a microfluidic channel. The pumping mechanism is actuated by repeated (e.g., once or multiple times) application of in-plane tensile strain. The in-plane strain can be due to the skin deformation during human movements for a skin-mounted wearable device and related applications.

### CROSS-REFERENCE TO RELATED APPLICATIONS

This application claims priority from U.S. Provisional Patent Application 63/527,791 filed Jul. 19, 2023, which is incorporated herein by reference.

### FIELD OF THE INVENTION

This invention relates to the microfluidic wearable devices and methods thereof.

### BACKGROUND OF THE INVENTION

Typically, human motion operated microfluidic pumps are limited to finger-triggered mechanisms. A microfluidic pump that can be configured to be operated by any human movement without an intentional decision from the user can open up new possibilities for wearable devices. Such a microfluidic device can be actuated using skin-strain. Skin-strain induces in-plain strain on skin-mountable wearable devices. In-plane tensile strain causes volumetric expansion in microfluidic channels. The art teaches microfluidic sensors based on volumetric expansion. These are sensors that detect strain or deformation by measuring changes in fluid displacement within a microfluidic channel or device. These sensors utilize the principle that when a strain or deformation is applied to a microfluidic system, it causes a change in the volume hence a displacement of the fluid within the system.

When a tensile strain or deformation is applied to these sensors, it causes the microfluidic channel or chamber to extend in strain direction and contract slightly in the perpendicular direction for standard elastomeric strain sensors that has positive Poisson's ratio in the range of 0 to 0.5. This causes a dilatation, positive volumetric strain change, hence negative pressure within the fluidic channel. In this way, the working liquid is pulled into the microfluidic channels when channels are subjected to a tensile strain in perpendicular direction to their elongation.

On the other hand, mechanical metamaterials can be utilized to achieve the opposite behavior. The mechanical metamaterials enable customized Poisson's ratios that are beyond the range of standard Poisson's ratio (0-0.5). Mechanical metamaterials when subjected to tensile strain, can exhibit lateral extension (Poisson's ratio <0) or contract significantly in lateral direction (Poisson's ratio >0.5) causing volume reduction rather than dilatation.

The present invention advances the art of microfluidic devices and pumps.

### SUMMARY OF THE INVENTION

The present invention provides a microfluidic device that has a negative volumetric strain under a tensile strain and is capable of pumping fluids stored inside a reservoir towards an outlet through a microfluidic channel. The pumping mechanism is actuated by repeated (e.g., once or multiple times) application of in-plane tensile strain. The in-plane strain can be due to the skin deformation during human movements for a skin-mounted wearable device and related applications.

In a first embodiment a microfluidic device is characterized by a first reservoir having a first patterned microfluidic channel. There is a first inlet at one end of the first patterned microfluidic channel, and a channel connecting to another end of the first patterned microfluidic channel. An outlet is connected to the channel which could be at a position along the channel or at an end of the channel. Strain applied to the first patterned microfluidic channel causes a first pumping mechanism of liquid flow from the first patterned microfluidic channel to the outlet via the channel. The first inlet can be used for filling one or more fluids (e.g. air, liquid, drugs and/or reagents) to the first patterned microfluidic channel during the first pumping mechanism. In another example, the first inlet can be sealed for a first reversible liquid displacement as opposed to the first pumping mechanism. In other words, the first inlet is a closed or an open inlet.

In a second embodiment, a microfluidic device is characterized by extending the first embodiment with a second reservoir having a second patterned microfluidic channel. There is a second inlet at one end of the second patterned microfluidic channel, and the channel now connecting to another end of the second patterned microfluidic channel. Strain applied to the second patterned microfluidic channel causes a second pumping mechanism of the one or more liquids flow from the second patterned microfluidic channel to the outlet via the channel. The first and the second inlet can be used for filling the one or more liquids to the second patterned microfluidic channel during the second pumping mechanism. In another example, the second can be sealed for a second reversible liquid displacement as opposed to the second pumping mechanism. In other words, the first and the second inlet are closed or open inlets.

The first and the second patterned microfluidic channel have a corrugated shape or a bell shape extending outwardly from a base of the channel. The channel has an aspect ratio for a height to a width (h_{base}:w)) of less than 1 (FIG. 1). An optimal aspect ratio is less than 1:3. A smaller aspect ratio by reducing the h_{base} is preferable to have a larger positive liquid displacement. As an example, a base channel with a width of 100 micrometers should have a base height of less than 30 micrometers for more efficient operation.

The first and the second patterned microfluidic channel have a membrane thickness in a range of 1 micrometer to 500 micrometers and a bump height, h, larger than 1 micrometer. In one example, the film thickness, t_{f} should be larger than the base channel height, h_{base}, and less than 2×h_{base}. An optimal thickness should be approximately 1.5×h_{base}. The t_{f} is critical to determine membrane thickness, tₘ as tₘ is equal to t_{f}−h_{base}. The exact value of tₘ is related to the bending stiffness of the membrane. The bell shape membrane should be stable and able to carry its own weight without any collapses when there is no strain. For efficient pumping, the bell shape membrane should buckle when strain is applied. For efficient pumping, the bell shape membrane should collapse to the channel floor and make contact with the channel floor immediately after strain is released. For efficient pumping, the bell-shape collapses should recover after strain is released.

The embodiments are interpreted as devices as well as methods for microfluidic devices and skin-mountable microfluidic devices.

The strain-driven microfluidic pump described herein offers several significant advantages over traditional devices and methods. One of the primary benefits of the embodiments is that they are power-free, eliminating the need for external power sources and thereby enhancing portability and usability in wearable applications. This makes the device thinner and more comfortable.

Finger-actuated pumping (FAP) is an alternative technology that relies on the deflection of an elastic membrane. FAP requires intentional application of finger force on a hemi-spherical membrane for actuation. This prevents continuous actuation (e.g., hundreds) over long periods of time (e.g., hours). FAP often suffer from variability due to inconsistent manual pressure, leading to less reliable fluid control. Furthermore, FAP require complex valve mechanisms to function properly, adding to their fabrication complexity and potential for malfunction. By leveraging the membrane deformation properties of elastomers, the presented embodiments achieve precise and consistent flow control through self-generated membrane collapses, eliminating the need for additional valve mechanisms.

The strain-driven pump introduced herein has one-step fabrication method utilizing the shrinkage of elastomers during thermal curing. This simplicity reduces production time and costs.

### BRIEF DESCRIPTION OF THE DRAWINGS

FIG. 1 shows according to an exemplary embodiment of the invention a schematic representation of the cross section of the out of surface microfluidic channel (OSMiC) reservoir.

FIGS. 2A-D show according to exemplary embodiments of the invention variations of the device with a top-down schematic representation of the device (left of each figure) and side view schematic of the device (right of each figure).

FIG. 3 shows according to an exemplary embodiment of the invention a top-down view schematic of the device with multiple reservoirs.

FIGS. 4A-B shows according to exemplary embodiments of the invention schematic representation of the fabrication procedures. FIG. 4A shows a 3D printing method: Illustration of the fabrication process using 3D printing technology. FIG. 4B shows Modified thermal bonding (mod-TB) method: The schematic representation of the cross-section of the OSMiC reservoir after spin coating PDMS (top) and after bonding on the partially cured (sticky) PDMS (middle). The bottom row shows the actual photos after PDMS spin coating (left) and after thermal bonding on sticky base (right). The critical parameters, tm and h represent membrane thickness and OSMiC height, respectively.

FIG. 5A-D shows according to exemplary embodiments of the invention the cross-section image of the out-of-structure microfluidic channels with thin membrane and oval base. FIG. 5A-B represent variations of OSMiC reservoir cross-sections with various channel heights. FIG. 5C shows reservoir without a bell shape structure (no bell shape). FIG. 5D shows connection channels.

FIG. 6 shows according to an exemplary embodiment of the invention the pumping performance comparison of microchannels with and without bell shape structure.

FIG. 7 shows according to an exemplary embodiment of the invention deformation characteristics of out-of-surface microfluidic channels with thin membrane and oval base during initial (no strain), strained, and after strain release conditions depending on strain percentages i.e. 5, 10, 15, 20%.

FIG. 8A-I shows according to exemplary embodiments of the invention snapshots of membrane deformation dynamics captured at 50-millisecond intervals from (FIG. 8A) to (FIG. 8I). In (FIG. 8A), the channel is under strain, while in (FIG. 8B), the strain begins to release. From (FIG. 8C) to (FIG. 8I), the strain is fully released. Note that there is a 4-second wait period between (FIG. 8H) and (FIG. 8I).

FIG. 9 shows according to an exemplary embodiment of the invention different pumping conditions: open pump, supply pump, supply reservoir pump. “Strain release” row shows the structure right after strain release and “Strain release*” row show the structure after reaching steady-state.

FIG. 10 shows according to an exemplary embodiment of the invention. Pumping performance of OP, SP, and SRP over fifteen strokes.

FIG. 11 shows according to an exemplary embodiment of the invention pumping performance comparison at different strain percentages over ten strokes.

FIG. 12A-B show according to exemplary embodiments of the invention pumping performances with in FIG. 12A the contact angle measurement of different liquids on PDMS composed of 50% water and 50% vegetable glycerin with 5% Tween-20 (orange), 50% water and 50% vegetable glycerin (green), 95% water and 5% vegetable glycerin. FIG. 12B shows pumping performance of liquids with 68° contact angle and 97° contact angle.

FIG. 13A-B show according to exemplary embodiments of the invention microfluidic channels integrated with absorbent materials for controlled and prolonged liquid exposure to the skin. (FIG. 13A) The amount of liquid retained by the tissue increases with the number of strain strokes applied (e.g., 5, 10, 20, 30 strokes). (FIG. 13B) The graph shows the extent of the red-colored area on the tissue corresponding to the number of strain strokes applied.

FIG. 14A-E show according to exemplary embodiments of the invention illustrations of device usage for both air and liquid inlet conditions.

FIG. 15A-D show according to exemplary embodiments of the invention image sequence representing the membrane deformation characteristics as a response to strain application in the case of both inlet and outlet are closed and sealed with tape. (FIG. 15A), represents the embodiment without the application of any strain. (FIG. 15B), represents the embodiment immediately after strain release (FIG. 15C), and waiting after strain release (FIG. 15D).

FIG. 16 shows according to an exemplary embodiment of the invention pumping performance comparison of different time dynamics over five strokes for the application and releasing the strain.

FIG. 17 shows according to an exemplary embodiment of the invention pumping performance comparison of different time dynamics over five strokes for the application and releasing the strain.

FIG. 18 shows according to an exemplary embodiment of the invention pumping performance comparison of different OSMiC reservoirs with air inlets and varying geometrical parameters.

FIG. 19A-C show according to exemplary embodiments of the invention the relationship between liquid displacement and strain percentage. The liquid used for the experiments is ionic liquid (IL). FIG. 19A: Different microchannel structures that have different membrane thickness and bump height. FIG. 19B: Liquid displacement and strain percentage relation. FIG. 19C: Time dynamics of liquid displacement as a response the application of various strain magnitudes. Orange (triangle) and blue (circle) represents liquid displacement during strain application and subsequent release, respectively.

FIG. 20 shows according to an exemplary embodiment of the invention the cross-section of the device without channel base (h_{base}=0).

FIG. 21 shows according to an exemplary embodiment of the invention the relationship between liquid displacement and strain percentage the conformal (with bell-shape) and non-conformal (no bell-shape) channel architectures that have different parameters.

FIG. 22 shows according to an exemplary embodiment of the invention the integration of a standard conventional microfluidic network and OSMiC to demonstrate the significance of a NOT operation.

FIG. 23A-B show according to exemplary embodiments of the invention strain-driven powerless mixing FIG. 23A: Representation of the working mechanism. FIG. 23B: Images from three repeated cycles of the procedure show color changes in the mixing chamber. Graph showing contrast value measurements for the active and inactive fluidic transport chambers, illustrating the effectiveness of the mixing process.

### DETAILED DESCRIPTION

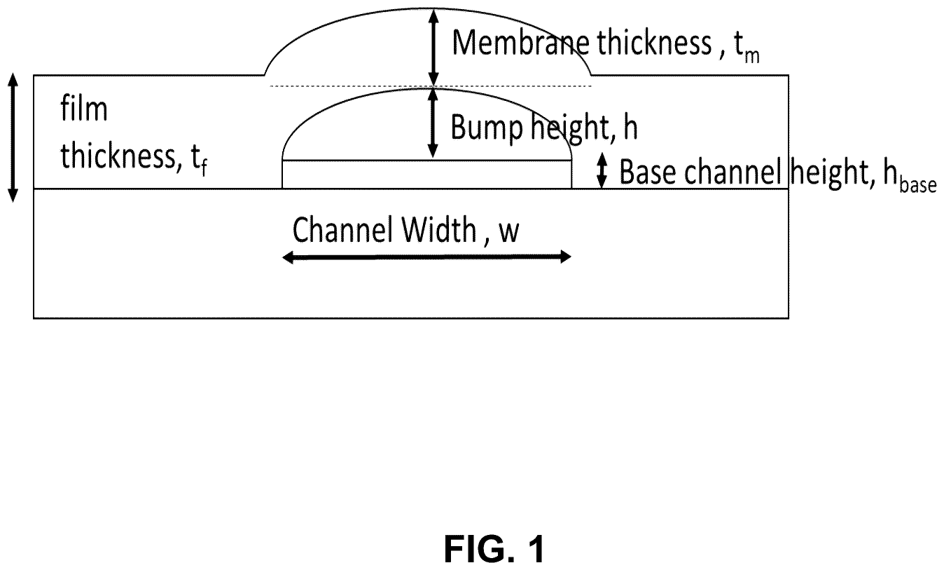

The present invention advances the art by patterning a microfluidic channel in a corrugated or bell shape. As such, a pumping (i.e., irreversible flow) towards the outlet or reversible liquid displacement (i.e., back and forth) can be achieved depending on the embodiment configuration, under repeated (e.g., once or multiple times) application of in-plane tensile strain. Reversible liquid displacement can be in the form of increasing liquid push for increasing strain or hybrid (i.e., first push and then pull for increasing strain). Using bell shaped reservoirs (called OSMiC) leads to negative volumetric strain within the fluidic channel that pushes the liquid when subjected to in-plane tensile strain. FIG. 1 shows a schematic representation of the cross section of the OSMiC reservoir. Different parameters that are shown here are studied and the results are analyzed.

By varying geometrical parameters of the microfluidic channels, such as bump height, h, base channel height. h_{base}, and membrane thickness, tₘ, liquid displacement can be characterized as pumping, reversible flow with push, or reversible flow with hybrid response (i.e., first pushing and then pulling as the applied strain is increased).

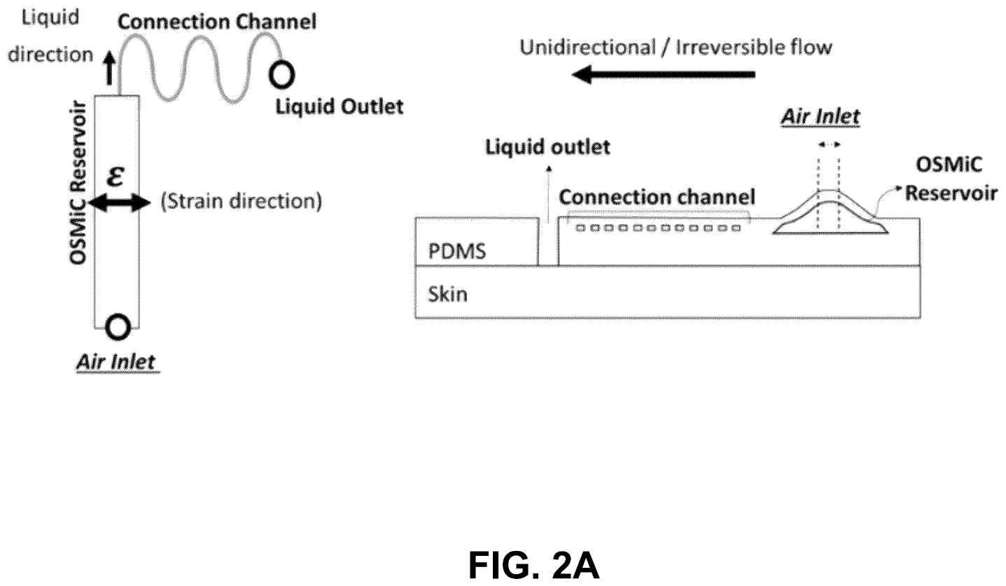

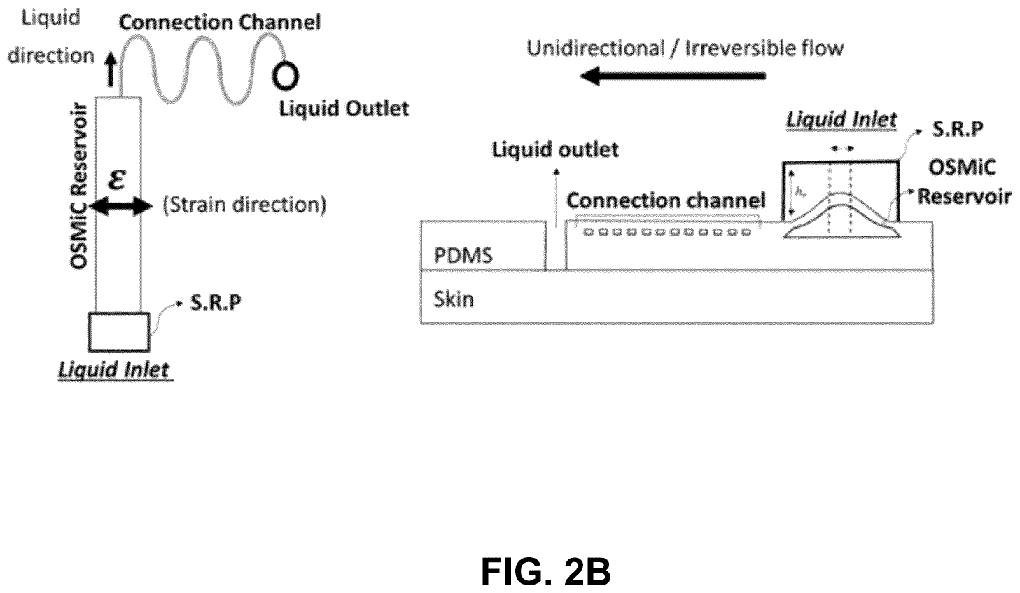

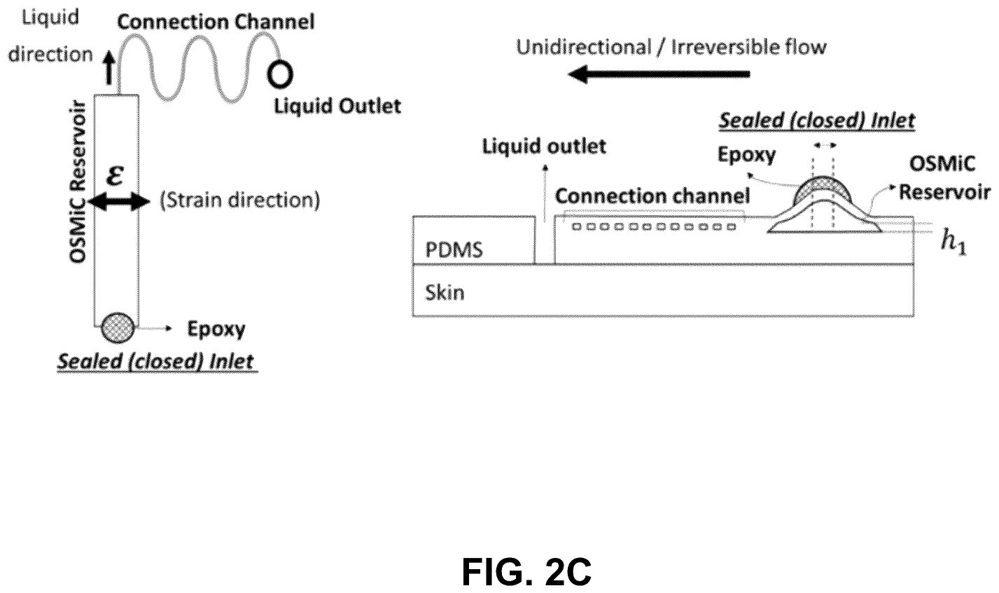

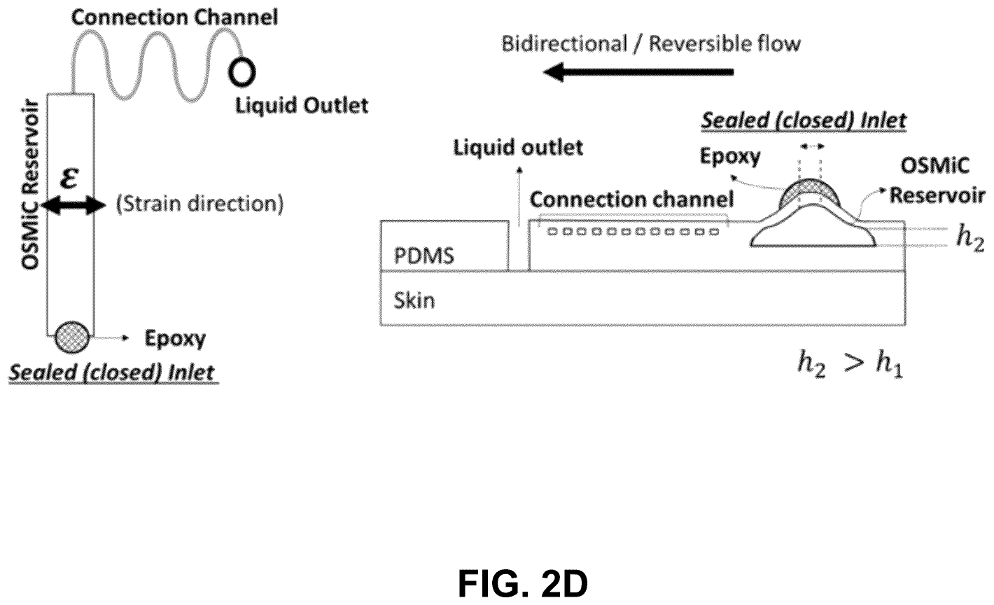

The overall device structure is shown in FIGS. 2A-D with variations to these geometrical parameters. FIGS. 2A-D represents the overall schematic of a first device embodiment. It has a reservoir (OSMiC), a connection channel, an inlet and an outlet. OSMiC is filled with liquid before the usage. Different types of liquid with different surface energies are compatible for the application (hydrophilic, hydrophobic or conductive liquid). First variation of the device shown in FIG. 2A where the OSMiC reservoir has small h_{base} (base channel height) between 0 and 100 micrometers and tₘ (membrane thickness) between 1 to 100 micrometers. The inlet and outlet are empty and connected to air. In FIG. 2B, conditions are same as FIG. 2A, but the inlet is connected to a supply reservoir that is filled with liquid and sealed. The outlet is connected to air. In FIG. 2AC the conditions are same, but the inlet is now sealed with an adhesive which can be an epoxy and the outlet is connected to air. FIG. 2D is a variation of the device in which h_{base} is larger than 100 micrometers. Here the inlet is sealed with adhesive and outlet is connected to air. Configurations shown in FIGS. 2A-B provide pumping towards the outlet in response to repeated in-plane tensile strain. FIG. 2C provide pumping towards the outlet in response to only a single in-plane tensile strain application. In FIGS. 2A-B, the pumping remains continuous from OSMiC to connection channel, and the total liquid displacement increases as strain is applied orthogonally to the OSMiC. In FIG. 2C, pumping occurs only once with the application of a single strain. Continuous application of strain beyond the maximum point does not result in any further liquid displacement. In FIG. 2D, no pumping is observed under the same conditions described in FIGS. 2A-B. However, when the inlet is sealed with an adhesive and strain is applied (similar to condition in FIG. 2C), the liquid is pushed from the reservoir into the connection channel. Upon releasing the strain, the liquid returns to its initial position, resulting in a reversible flow. If the strain is increased sufficiently, the liquid initially pushed by smaller strains begins to be pulled back, resulting in less overall liquid displacement for a single strain stroke. This behavior is described as hybrid liquid displacement, involving both pushing and pulling of the liquid.

Pumping described above can be used for several applications such as lab-on-a-chip devices for accurate and control movement of reagent and samples, biomedical diagnostics including blood test, pathogen detection and biomarker analysis, drug delivery system for controlled unidirectional flow and precise dosing, wound healing combination with drug delivery for localized treatment and sterilization, chemical synthesis, and environmental monitoring like detection of pollutants and toxins, and sweat sensing. On the other hand, reversible flow can be used in physiological biosensing applications for human motion detection and monitoring where the controlled fluidic movement induced by reverse displacement can be utilized for sensing and tracking purposes. It can also be used with the combination of pumping components to obtain integrated microfluidic systems for chemical synthesis and analysis and multiple reaction steps.

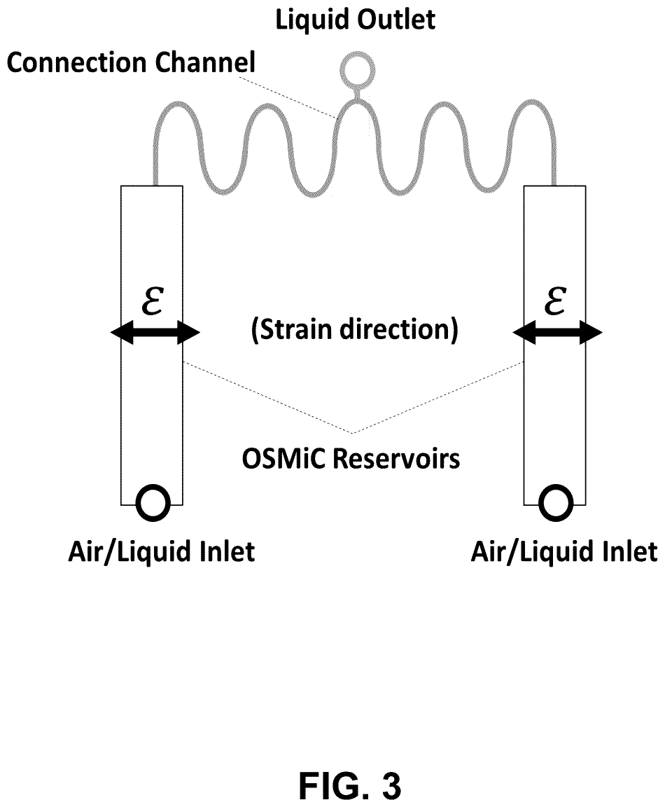

To increase the pumping performance and to create integrated strain sensor structure, it is possible to combine multiple reservoirs as shown in FIG. 3. Electrodes can be connected to two ends of the connection channel for strain sensing applications.

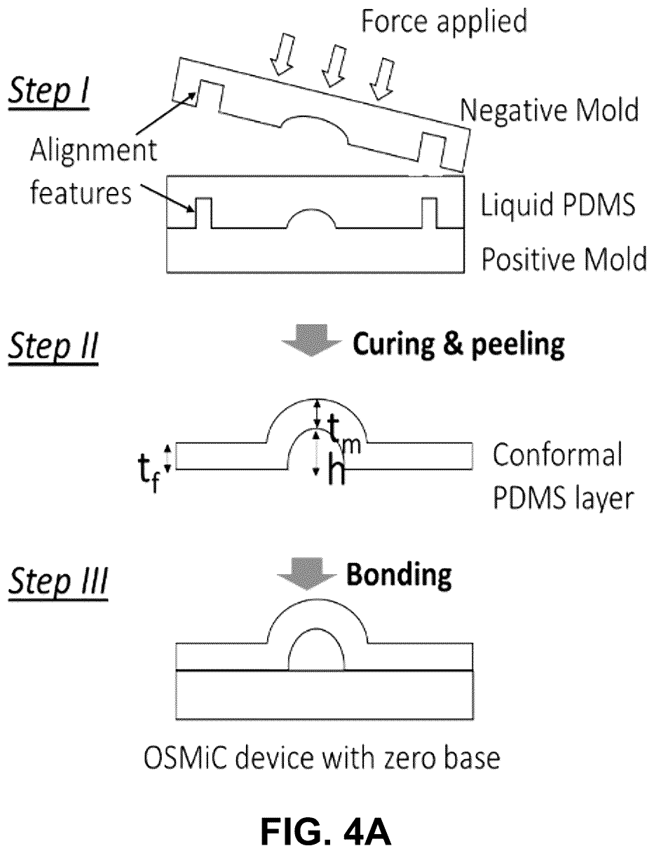

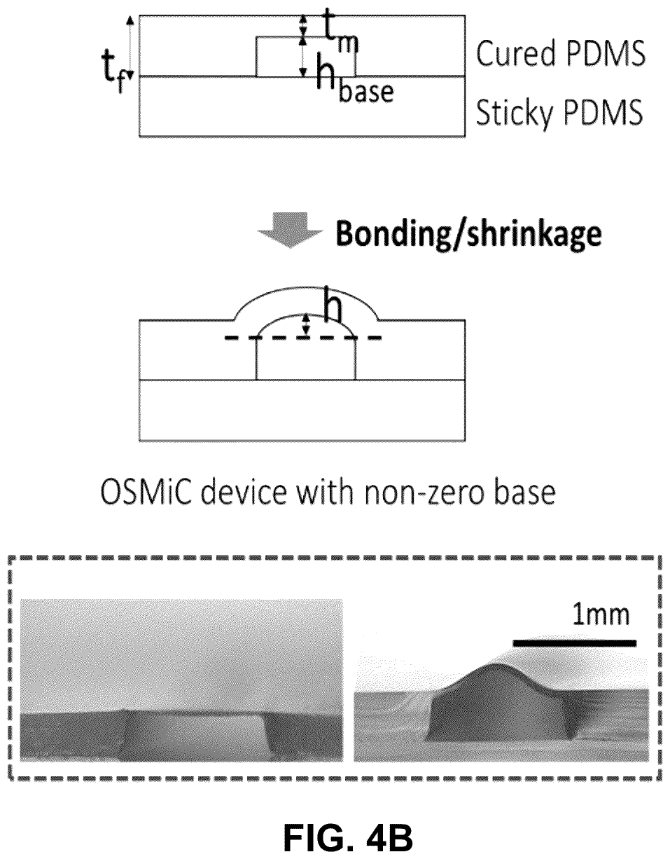

The devices can be fabricated using two exemplary techniques as shown in FIGS. 4A-B. FIG. 4A illustrates a 3D printing method. In this method, a CAD model is used to design a two-piece mold that has top and bottom parts. The design is then transferred to the stereolithography (SLA) 3D printer. After printing, molds are washed with isopropyl alcohol (IPA) for 20 minutes, cured under UV using the time function of the UVC-1000 device (Hoefer Inc.) at maximum energy setting for an hour, and baked in at 80° C. oven overnight. The bottom part of the mold is placed in a petri dish (positive mold) and a 10:1 ratio of PDMS is poured. Before placing the top part (negative mold), the PDMS is degassed in a vacuum for 30 minutes to eliminate bubbles. After the top part is placed on to the bottom and the two pieces are clicked using a post-hole system, the piece is baked in the oven at 80° C. for about an hour to have cured patterned PDMS sandwiched between the two parts. The fully cured PDMS is removed and inlets and outlets are punched. Then it is placed on the partially-cured sticky PDMS that was prepared using a 20:1 ratio PDMS spin-coated on a blank silicon wafer, and baked for 10 minutes at 80° C. This partially cured bottom piece provides bonding and eliminates contact problems due to the surface roughness of the sample that is made using 3D printing. The complete device is then put into the oven for baking for 2 hours at 80° C. After baking, reservoirs are filled with the desired working liquid. Using this fabrication method, OSMiC with no base (h_{base}=0) can be fabricated.

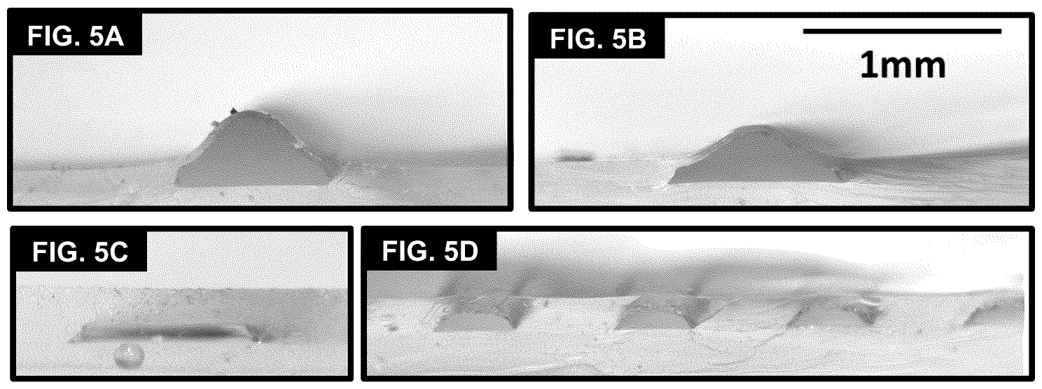

FIG. 4B illustrates the application of a modified thermal bonding (mod-TB) method, which utilizes the PDMS shrinkage as the basis of OSMIC creation. Typically, PDMS shrinkage is considered undesirable and can lead to issues such as misalignment and leakage. To address these challenges, researchers have extensively investigated the shrinkage behavior of PDMS under various mixing ratios and curing conditions. For the purposes of this invention, the inventors used PDMS shrinkage as a mechanism for generating OSMiC structures from different types of molds including those produced through photolithography techniques. This eliminates the feature size limitation of the 3D-printing method and provides a versatile fabrication method for the skin-mountable devices that are required to be thin and compatible with the skin mechanical load. The shrinkage-driven OSMiC structures were achieved through the manipulation of PDMS ratios, spin speeds, and control of baking time. PDMS is poured/spin-coated on the mold that has microchannel patterns and depending on the desired final thickness, the spin speed is adjusted. Here the initial mold features a reservoir with a rectangular cross-section. PDMS is poured onto the molds and spin-coated at a controlled speed to achieve the desired membrane thickness in the range of 1 to 100 micrometers, optimally at 40 micrometers, then fully cured in an oven for about an hour. After fully curing, the PDMS is peeled off the mold and carefully placed onto the partially cured base. The combined piece is then returned to the oven for an hour to achieve a fully cured PDMS structure. This modified thermal bonding procedure transforms the initially rectangular reservoir into an out-of-surface bell-shaped structure, as seen in FIGS. 5a and 5b. Adjusting the partial curing time allows for control over the height of the bell shape. The optimal partial curing time for the base varies with oven temperature; lower temperatures require longer curing times.

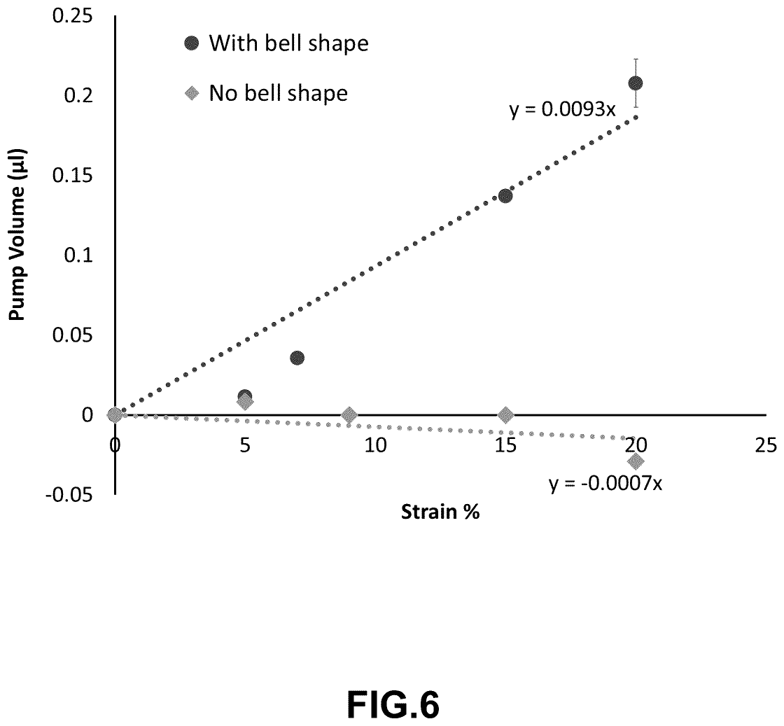

FIGS. 5A-D show cross-section images of the out-of-structure microfluidic channels with thin membrane and oval base. FIGS. 5A-B represent variations of OSMiC reservoir cross-sections with various channel heights. FIG. 5C shows reservoir without a bell shape structure (no bell shape). FIG. 5D shows connection channels. The described out-of-surface bell-shaped structure is crucial for the device's operation. Without it, the device does not exhibit unidirectional flow, as shown in FIG. 6. The structure without the bell-shaped channel (non-OSMiC) can be seen in FIG. 5C. The schematic representation of the cross-section of the OSMiC reservoir during the two stages of the fabrication and necessary parameters for obtaining pumping is described in FIG. 4B. To obtain continuous pumping for continuous application of periodic strain, membrane thickness, tₘ, should be in the range of 1 to 100 micrometers and OSMiC height, h, should be larger than 1 micrometer. The OSMiC reservoir channel width, w, has to be in the range of 1 micrometer to 10 millimeters. h_{base} has to be in the range of 0 to w, optimally less than w/3, optimally less than w/5. FIG. 6 shows the pumping performance comparison of microchannels with and without bell shape structure.

### Working Mechanism for Unidirectional Flow

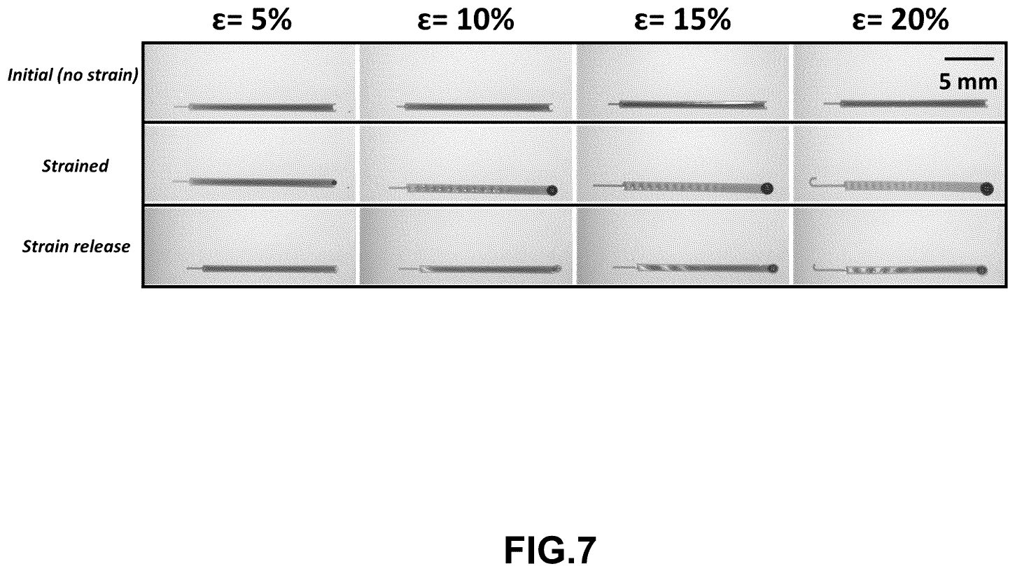

By introducing a thin (1 micrometers to 100 micrometers) membrane characteristic to the out-of-surface bell shape structure, the membrane deformations on the reservoir are created during the strain application and release as seen in FIG. 7. Here, it can be seen that larger strain causes larger liquid displacement and as a result achieving higher pumping rates is possible.

When strain is applied, the flexible membrane of the reservoir deforms, reducing its volume (10, 15, 20% strain). This deformation increases the pressure inside the reservoir. The pressure differential drives the liquid from the reservoir towards the outlet, overcoming the hydraulic resistance of the connection channel. Simultaneously small volume of liquid is pushed towards inlet. The tensile strain orthogonal to the OSMiC elongation causes a compression of the membrane in the parallel direction to OSMiC elongation due to the positive Poisson's ratio of the PDMS. This compression causes periodic membrane deformations (i.e., buckling) as seen in the middle row of FIG. 7. FIG. 7 shows the deformation characteristics of out-of-surface microfluidic channels with thin membrane and oval base during initial (no strain), strained, and after strain release conditions depending on strain percentages i.e. 5, 10, 15, 20%.

Upon releasing the strain, the volume in the OSMiC reservoir increases and pressure decreases. The reservoir membrane collapses due to the sudden drop in pressure. The membrane collapse stops backflow from connection channel to the reservoir. Following the strain release, the volume of the reservoir increases. Negative pressure pulls air or liquid, depending on the fluid present at the inlet. The inlet side offers less resistance compared to the connection channel therefore fluid enters from the inlet side instead of outlet side. As air or liquid is drawn into the reservoir from the inlet, the membrane pops back to its original position starting from the inlet side and moving towards the outlet side (e.g., right to left) (FIG. 8C-G). This right to left (from inlet to outlet direction) recovery of the collapses is an action that further drives the liquid from the inlet to the outlet similar to the peristaltic pumping. The temporary collapse and subsequent popping back of the reservoir membrane creates flow durations to drive the liquid in one direction, ensuring unidirectional flow from the reservoir to the connection channel.

### Variations and Performance

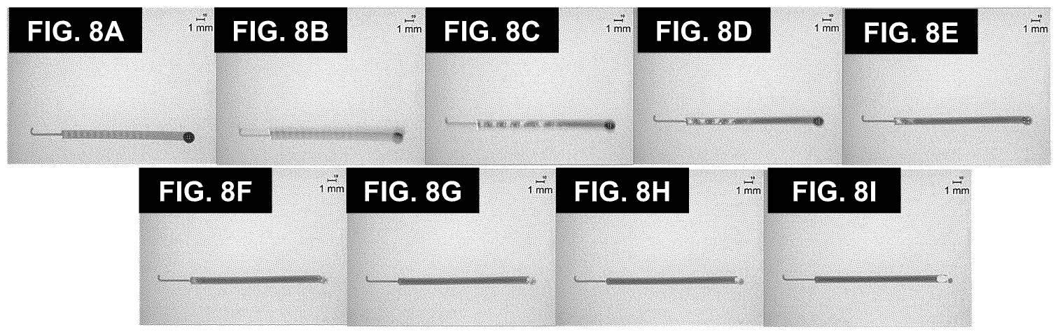

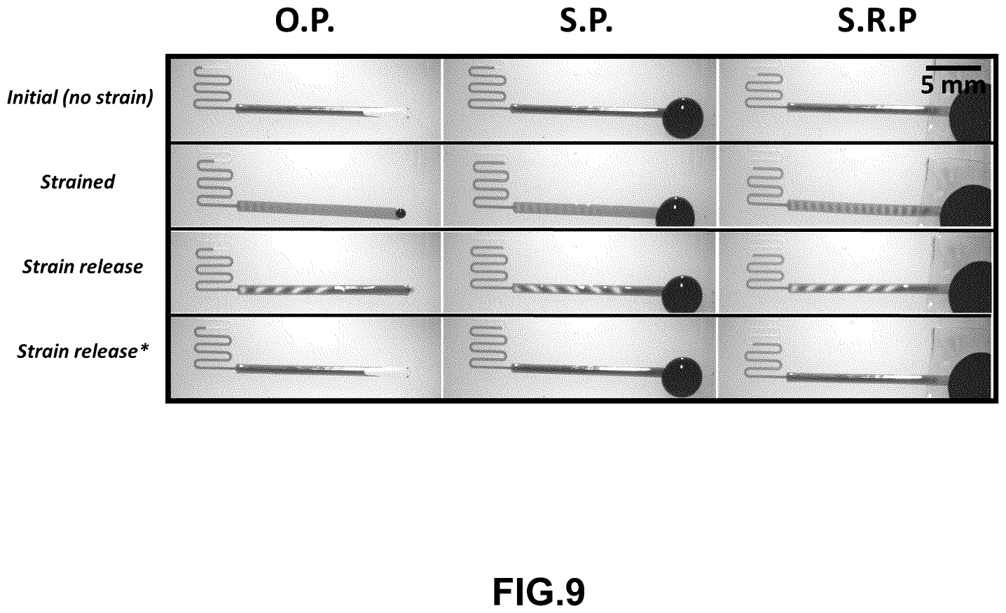

The structure of the microfluidic is compatible with various conditions, as demonstrated in FIG. 9. In FIG. 9 different pumping conditions are shown:
Open Pump (OP): Both outlets are open to the air.
Supply Pump (SP): The reservoir's outlet is filled with excess liquid.
Supply Reservoir Pump (SRP): An additional reservoir made of PDMS is created, filled with liquid, sealed using tape, and then attached to the channel's reservoir outlet.

“Strain release” row shows the structure right after strain release and “Strain release*” row show the structure after reaching steady-state.

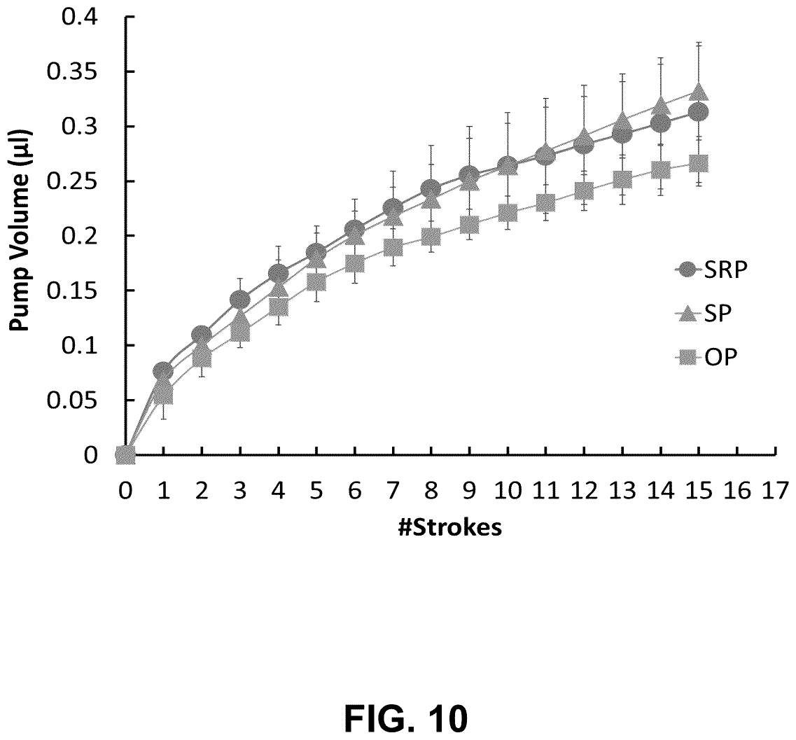

Pumping performance of OP, SP, and SRP over fifteen strokes are presented in FIG. 10.

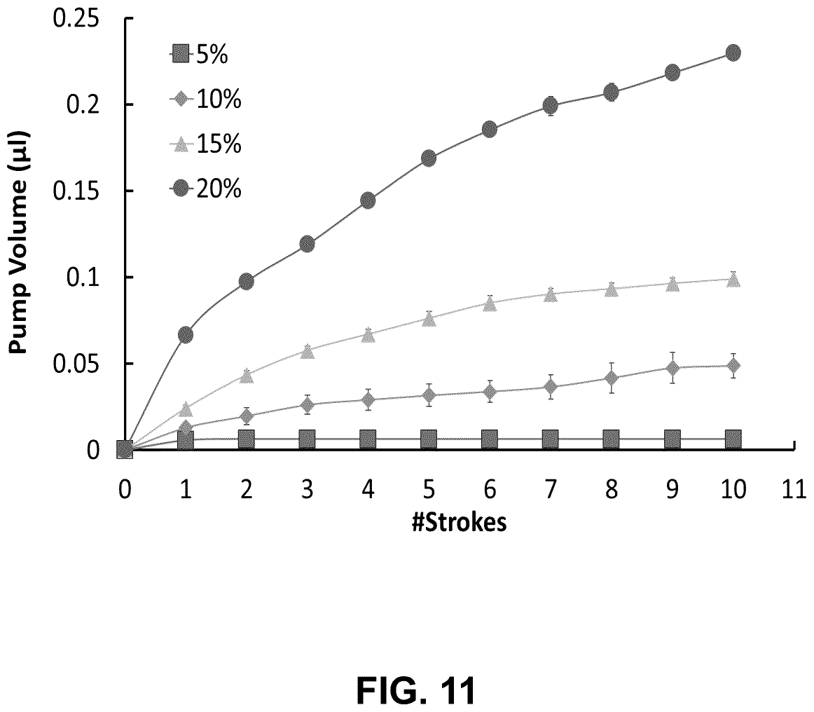

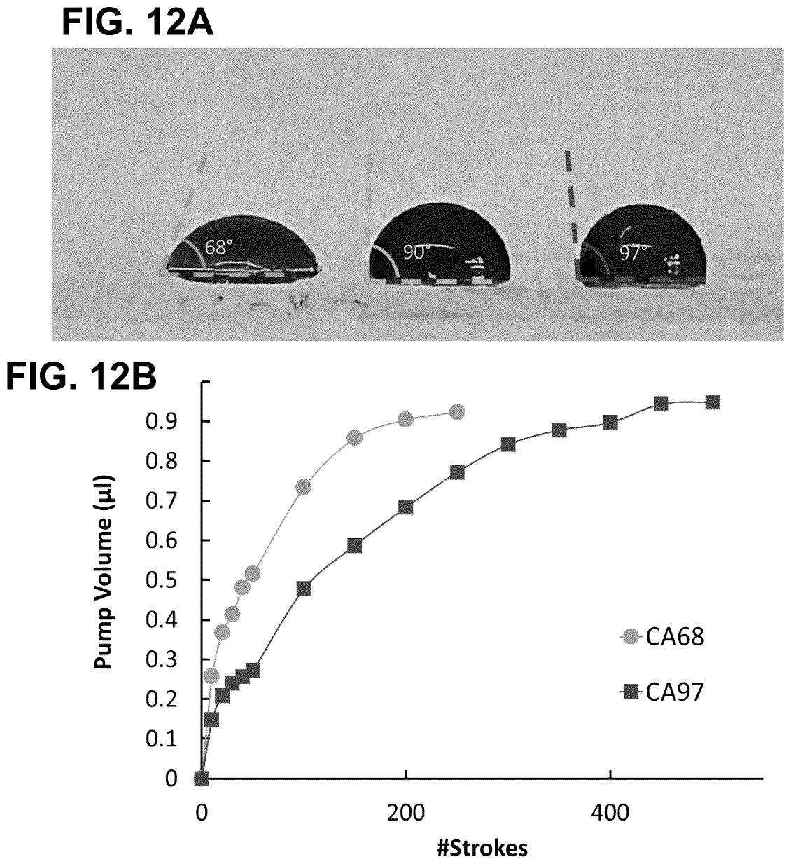

FIG. 10. Pumping performance of OP, SP, and SRP over fifteen strokes. As shown in FIG. 11, the pumping performance improves with increasing strain magnitude. The channels can accommodate various liquids with different contact angles, such as hydrophilic and hydrophobic liquids, as demonstrated in FIGS. 12A-B. FIG. 12A shows the contact angle measurement of different liquids on PDMS composed of 50% water and 50% vegetable glycerin with 5% Tween-20 (orange-left), 50% water and 50% vegetable glycerin (green-middle), 95% water and 5% vegetable glycerin (blue-right). FIG. 12B shows pumping performance of liquids with 68° contact angle and 97° contact angle.

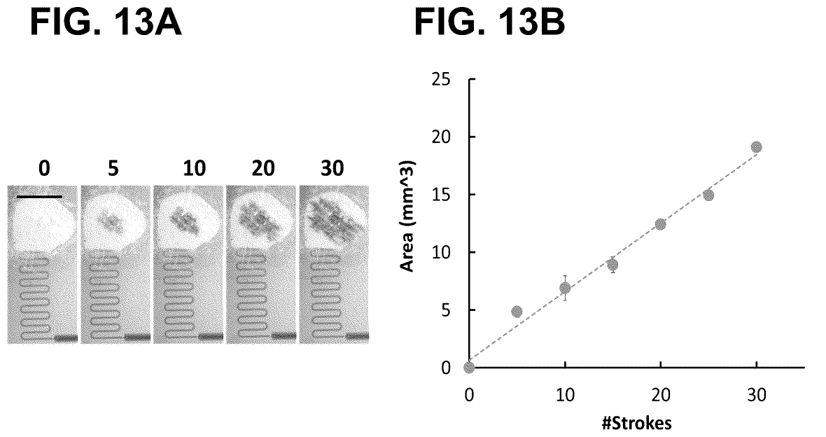

Combining microfluidic channels with absorbent materials such as paper or hydrogel to create an interface with the skin can provide controlled and prolonged exposure to the liquid. FIGS. 13A-B illustrate this setup, which shows promise for drug delivery and wound healing applications. The design of the microfluidic system ensures that liquid is moved in a controlled manner from the reservoir to the connection channels, delivering a precise volume. The absorbent material holds the liquid expelled from the connection channel's outlet, enabling a controlled release onto the skin. The porous structure of the absorbent tissue retains the liquid and releases it slowly over time, ensuring prolonged exposure. Additionally, as the number of strain strokes increases, more liquid is held by the absorbent material, allowing for potential dosage adjustments.

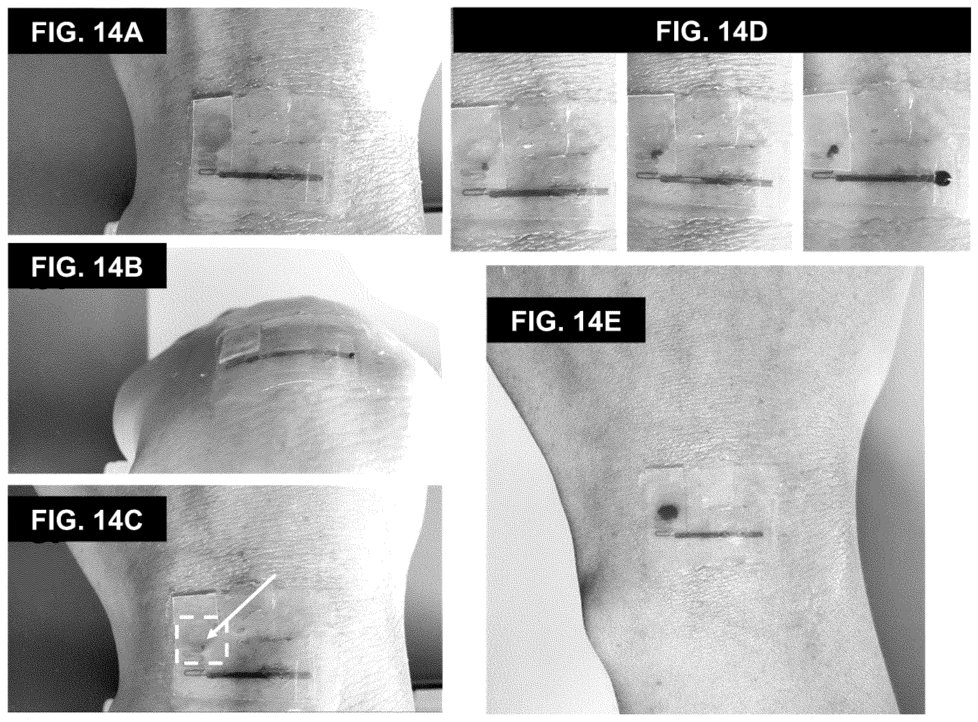

FIGS. 14A-E illustrate the device in use on a human wrist. The volunteer extended their wrist to create strain and membrane deformations, then released and repeated the movement to pump liquid out of the OSMiC reservoir. FIGS. 14A-E show illustrations of device usage for both air and liquid inlet conditions. FIG. 14A is where the device is initially filled with liquid and has an air inlet. FIG. 14B shows where a volunteer extends his/her wrist, creating unidirectional flow. FIG. 14C shows upon wrist relaxation, the liquid is pumped out of the device. FIG. 14D shows liquid collection at the outlet is shown after five repetitions of wrist extension and relaxation (left). After twenty repetitions, membrane collapse became irreversible under air inlet conditions (middle). Introducing additional liquid from the inlet restored the membrane collapses (right). FIG. 14E shows after fifty repetitions of wrist extension and relaxation, further liquid collection is observed.

### Control Experiments

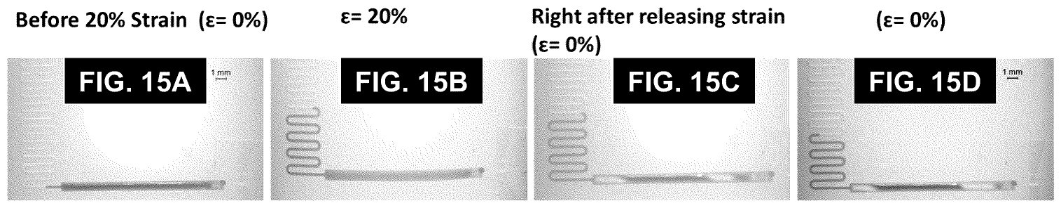

Control experiments were performed in the case of both inlet and outlet are closed and sealed with tape (FIGS. 15A-D). When strain is applied, the flexible membrane of the reservoir deforms, reducing its volume. The deformation increases the pressure inside the reservoir. Since the inlet and outlet are closed, the fluid cannot escape through them, causing an increase in internal pressure. The pressure differential forces the liquid to move from the reservoir into the connection channels, which have a volume to accommodate the excess liquid. Upon releasing the strain, the pressure inside the reservoir decreases as the volume of the reservoir increases. The sudden drop in pressure causes the membrane to collapse, generating a negative pressure impulse. (FIG. 15C). This negative pressure impulse would typically stop backflow and pull liquid or air from the nearest outlet if they were open. However, with both outlets closed, the fluid cannot be pulled from outside the reservoir. Since no additional liquid or air can enter the reservoir from the outlets, the liquid from the connection channel draw back to reservoir. The liquid drawn back from the connection channels into the reservoir does not fully return because the membrane remains collapsed. The membrane's elasticity tries to return it to its original shape, but the lack of external pressure support prevents this from happening. In this case, the inability to equalize pressure with the external environment is the key factor preventing the membrane from popping back and achieving unidirectional flow (FIG. 15D).

### Time Dynamics of Strain Application and Release:

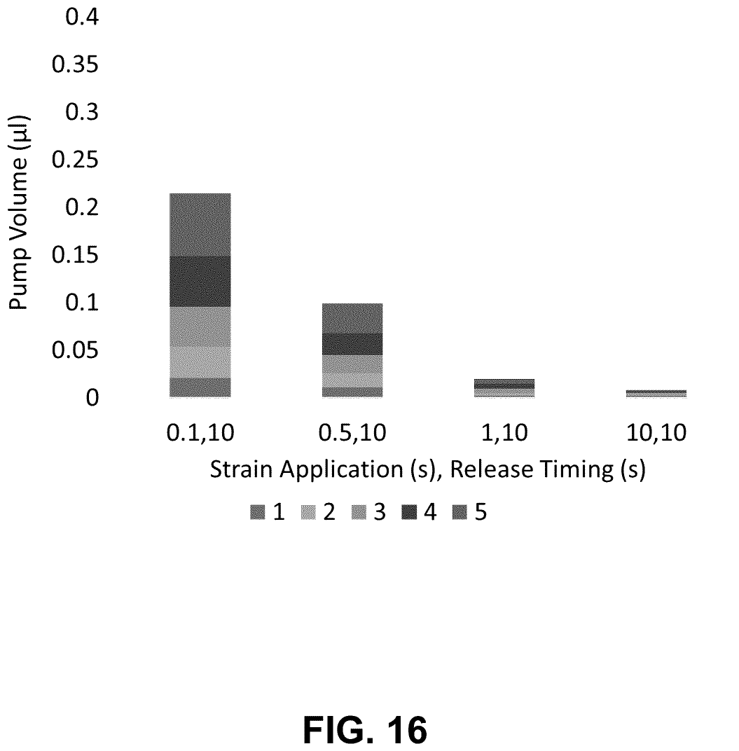

The pumping performance varies based on the timing of strain application and release. FIG. 16 shows pumping volume results for 5 different strain application times (0.1 s, 0.5 s, 1 s, and 10 s) of five strokes; each stroke consists of strain application and subsequent release. It is seen that rapid application of the strain is critical in achieving higher pumping rate. When strain is applied quickly, the flexible membrane of the reservoir deforms rapidly. This quick deformation results in a sudden decrease in the volume of the reservoir. The rapid volume reduction creates a sharp and significant increase in internal pressure within the reservoir. The high-pressure spike generated can overcome the resistance of the connection channels more effectively. The sudden push results in a larger volume of liquid being displaced from the reservoir into the connection channels. When strain is applied slowly, the membrane deforms gradually, leading to a more gradual decrease in the reservoir's volume. The slower deformation results in a more moderate increase in internal pressure. The moderate pressure increase may not be as effective in overcoming the high resistance of the connection channels. Leading the volume of liquid displaced from the reservoir to the connection channels is being smaller compared to fast strain application.

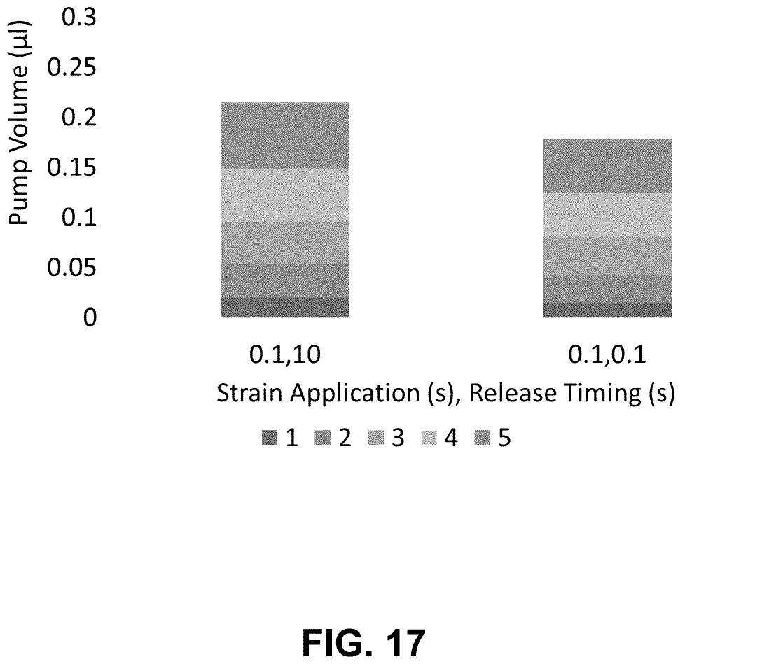

FIG. 17 compares the pumping performance across different time dynamics for two different strain release times (10 s vs 0.1 s) over five strokes. It is shown that, releasing the strain at different speeds does not have a significant impact on the pump efficiency.

Importance of Occurrence of Membrane Collapses and Effect of Height of Base Channel (h_{base})

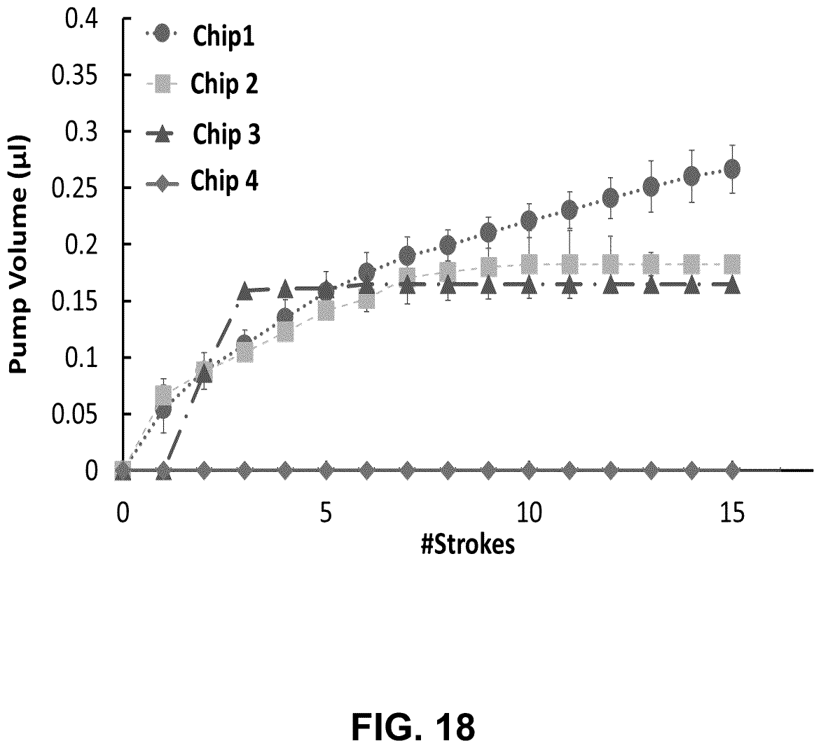

The experiments were conducted using OSMiC reservoirs with air inlets and varying geometrical parameters. Chip 1 and Chip 2 both feature 800 micrometers width and a 60 micrometers base channel height. Chip 1 has a 40 micrometers membrane thickness, while Chip 2 has a 70 micrometers membrane thickness. As depicted in FIG. 18, thicker membranes where collapse is not visible do not sustain continuous pumping with repeated strain application and release.

Chip 3 has a width of 1.5 millimeters and a membrane thickness of 300 micrometers. It lacks a base channel (h=0). Due to the absence of membrane collapse during strain application, it does not sustain continuous pumping after the fourth strain application. Chip 4 has a width of 1 millimeter, membrane thickness of 40 micrometers and base channel height of 250 micrometers. Despite having the same membrane thickness as Chip 1, it does not pump at all due to the absence of membrane collapses caused by the larger base channel height. It is seen that thin membrane thickness and small base height provides membrane collapses and these collapses are critical in achieving continuous pumping.

Results for Configuration in FIG. 2D. (Reversible Flow and h_{base}≠0)

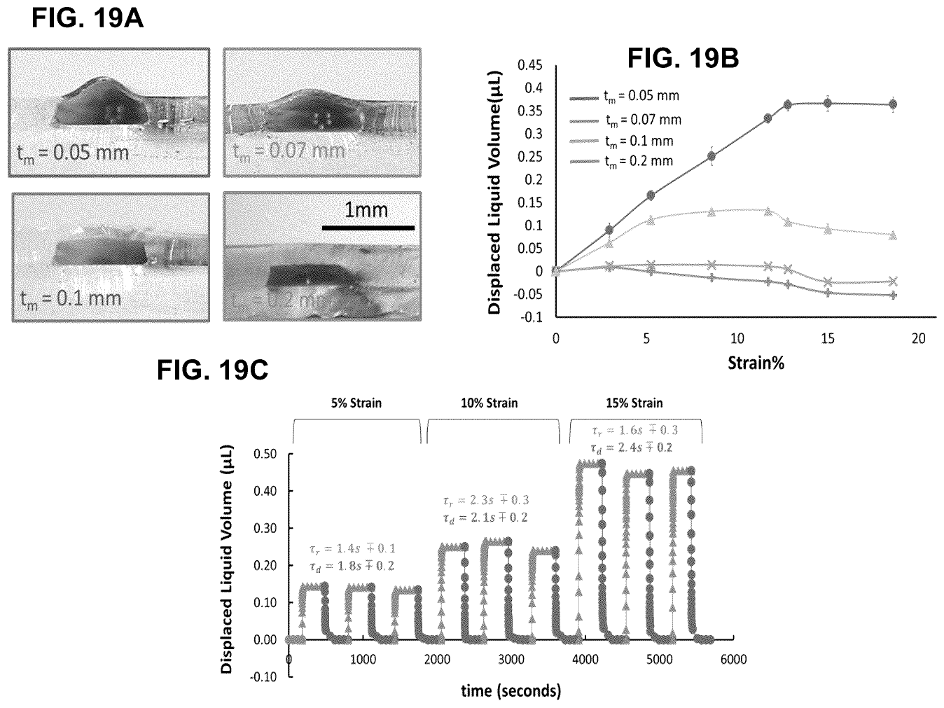

FIGS. 19A-C show results from the four different microchannel structures that have different parameters introduced in FIG. 1. These microchannels are fabricated using a modified version of a standard soft lithography method. The channel base height is 250 micrometers. A mold is used to create channels and polydimethylsiloxane (PDMS) is spin-coated on the mold. Thus, the membrane thickness is determined by the spin-coated film thickness, tr. The bell shape is formed during thermal bonding. When there is no bell shape (i.e., bump height is zero), the liquid displacement exhibits negative values. This is attributed to the positive volumetric strain experienced along the channel as a result of the deformation. Due to volumetric expansion, reservoirs pull air from the outlet, causing a subsequent pulling effect on the liquid, resulting in the observed negative values of displacement.

This invention highlights the effect of a bell-shaped membrane for having a negative volumetric strain values along the same microchannel as the strain is induced. This causes, for smaller strain values, liquid displacement to be reversed and the air to be pushed toward the outlet. For larger strain values, the bump height gets smaller and flatten at some point which causes positive volumetric strain along the rectangular part of the microchannel. The resultant air pull causes a fluid displacement in the negative direction (pull) after 10% strain as shown in FIG. 19D.

FIGS. 19A-C illustrate the relationship between strain and liquid displacement for different bump height and membrane thickness values. It has been observed that as the bump height increases, the positive liquid displacement increases. For increasing the bump height there is a need for smaller membrane thickness.

The inventors identified that the channels should have an aspect ratio (height to width (h_{base}:w)) of less than 1. The optimal aspect ratio is less than 1:3. A smaller aspect ratio by reducing the h_{base} is preferable to have a larger positive liquid displacement. As an example, a base channel with a width of 100 micrometers should have a height of less than 30 micrometers. The spin-coated film thickness, t_{f} should be larger than the base channel height, h_{base}, and less than 2×h_{base}. An optimal thickness should be approximately 1.5×h_{base}. The exact value is related to the bending stiffness of the membrane.

Results for Configuration in FIG. 2D. (Reversible Flow and h_{base}=0)

This device has no channel base (h_{base}=0). Here the bell shape of the membrane creates a round channel. In this device, membrane thickness and the film thickness can be varied separately, which provides a wider range of structures.

In this device, membrane thickness and film thickness can be varied provided by the flexibility of the 3D printing fabrication method the inventors have adopted. This allows the fabrication of a wider range of geometries. Here to make the microfluidic channels, two-piece molds that clicked to each other are printed and polydimethylsiloxane (PDMS) is poured between the molds to obtain various conformal structures.

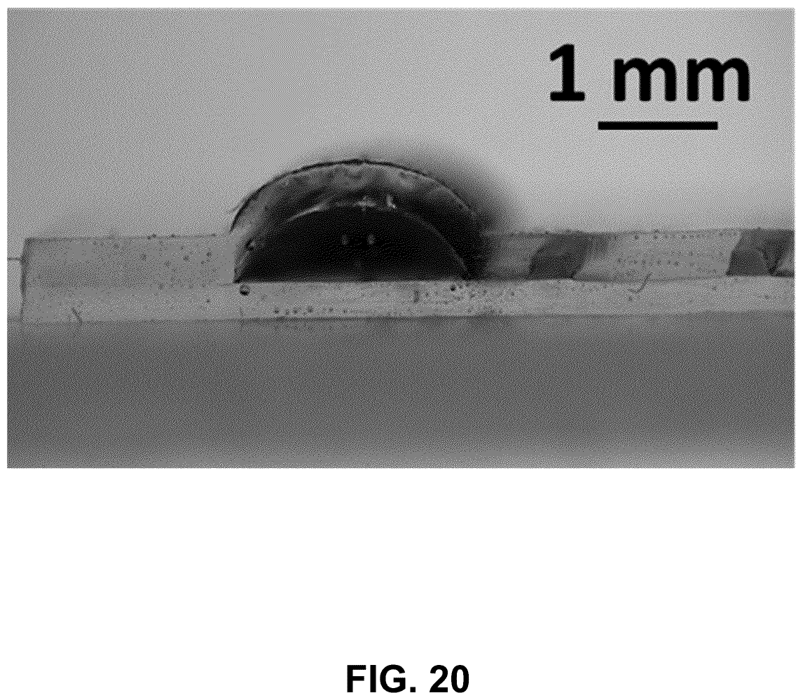

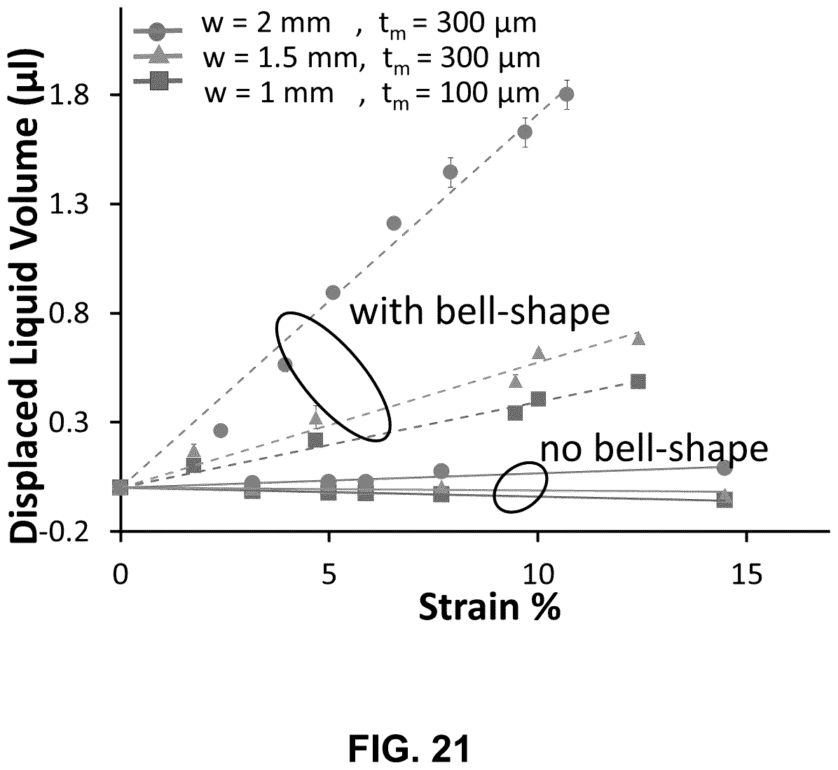

FIG. 20 represents the cross-section of device without channel base. Different parameters that are shown in FIG. 1 are studied and the results are analyzed. Here the inventors identified that the no-base, conformal devices shown in FIG. 20 exhibit negative volumetric strain as the strain is induced in the orthogonal direction to the sensor as shown in FIG. 2A. This causes the liquid displacement to be reversed and the air to be pushed toward the outlet. The no-base conformal devices do not show the hybrid response we have observed for device with base channel (FIG. 19) and push the air in a large range of strain values as shown in FIG. 21. Here the inventors identified that as the channel width increases the liquid displacement increases for the same bump height, h.

The film thickness should be equal to or smaller than 1.5 times the membrane thickness. The membrane thickness should be less than two times the bump height and it should be less than the bump height for more efficient operation.

The importance of the aspect ratio of the channel is also highlighted in FIG. 21 for the conformal channel architecture. All three channels have a bump height of 500 micrometers. However, the width of the microchannel is different for each case. For 1, 1.5, and 2 millimeters wide microchannels, the aspect ratio (h:w) is 1:2, 1:3, and 1:4, respectively. As the aspect ratio decreases, the total volume inside increases and the reverse fluidic displacement along the microchannel is increased which caused more liquid to be pushed out towards the outlet.

### Variations of Applications

The reverse fluidic displacement invention presented herein has a wide range of applications, making it highly versatile and valuable across various fields. One notable application lies in the detection of human movement, where the controlled fluidic movement induced by reverse displacement can be utilized for motion sensing and tracking purposes. Additionally, this invention holds promise in the field of drug delivery, enabling precise control over the movement and distribution of therapeutic substances within a biological system.

Furthermore, the potential combinations of different reverse and hybrid configurations offer even more possibilities. By integrating various reverse displacement mechanisms and hybrid setups, this invention can be utilized for pumping applications, facilitating the controlled transport of fluids within microchannels. Additionally, it can be employed in bioreaction analysis, enabling precise manipulation and monitoring of biochemical processes in a controlled fluidic environment.

In addition to the aforementioned applications, the reverse fluidic displacement invention can be effectively integrated with different types of microneedle systems such as solid microneedles, hollow microneedles, or biodegradable microneedles, further enhancing its potential for drug delivery applications. The integration of this innovation with microneedles allows for precise and controlled administration of therapeutic substances through the skin.

Furthermore, dissolving or biodegradable microneedles are designed to dissolve or degrade within the skin, releasing the drug payload in a controlled manner. This type of microneedle eliminates the need for needle removal after administration and offers a convenient and safe drug delivery method.

By integrating the reverse fluidic displacement invention with different microneedle systems, it becomes possible to precisely control the flow and distribution of drugs or substances through the microneedles. The reverse displacement mechanism can be harnessed to enhance drug release, increase delivery efficiency, and enable targeted delivery to specific skin layers or tissues.

This integration opens up new possibilities for advanced drug delivery applications, providing a platform for innovative and tailored therapeutic approaches. The combination of the reverse fluidic displacement invention with various microneedle types offers a comprehensive and versatile solution for controlled and efficient drug delivery, with potential benefits in areas such as transdermal therapy, vaccination, and personalized medicine.

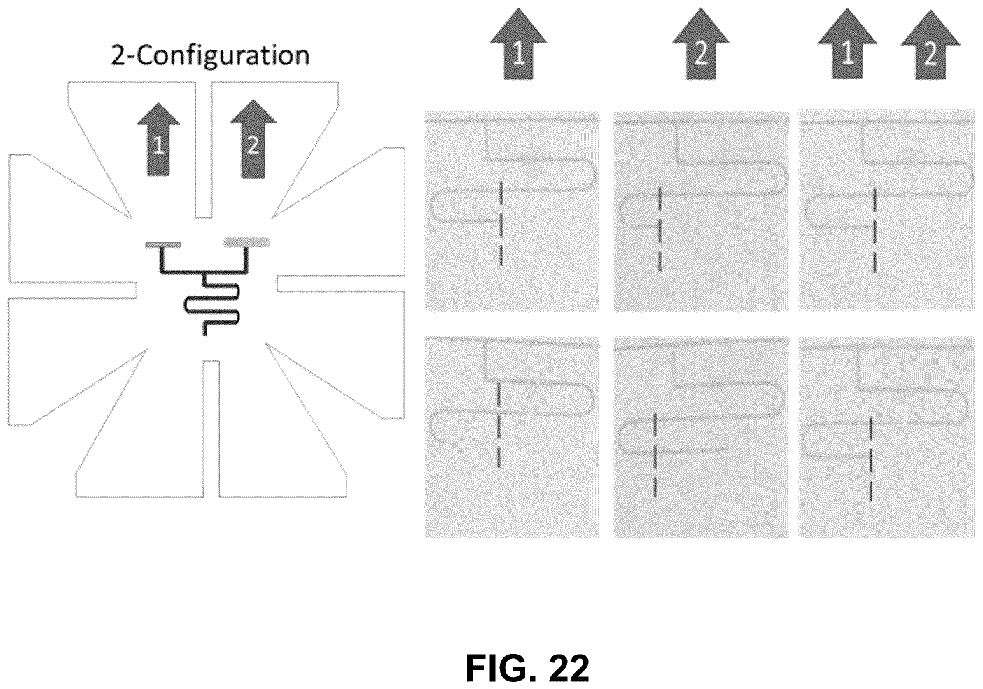

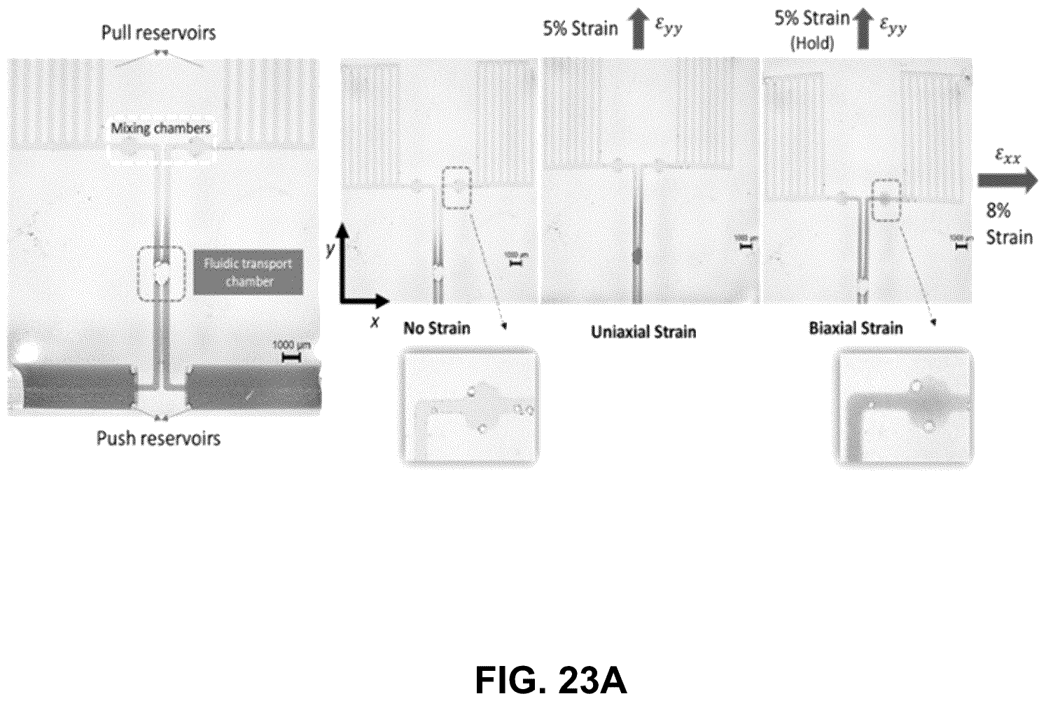

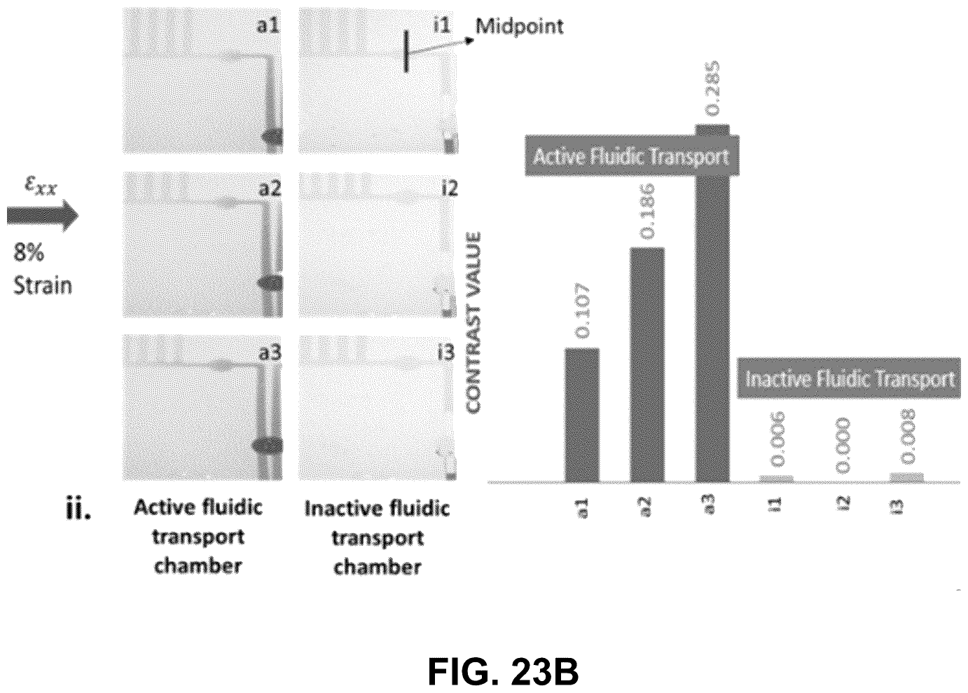

Inventors have developed devices which have been integrated for different operations such as wearable sensors for movement detection (FIG. 22), drug delivery (FIG. 14A-E) and mixing as a bioreaction application (FIGS. 23A-B). Further characterization for multiple channel cases and different configurations of sensors can be combined to exhibit the potential of reverse and hybrid liquid displacement for various applications.

A standard pulling microfluidic network and reverse pushing microfluidic network can be integrated together to achieve complex functions. The inventors have demonstrated that they can cancel each other's effect (FIG. 22).

What is claimed is:

1. A microfluidic device, comprising:
(a) a first reservoir having a first patterned microfluidic channel; and
(b) a first inlet at one end of the first patterned microfluidic channel;
(c) a channel connecting to another end of the first patterned microfluidic channel; and
(d) an outlet connected to the channel,
where strain applied to the first patterned microfluidic channel causes a first pumping mechanism of liquid flow from the first patterned microfluidic channel to the outlet via the channel, and
wherein the first inlet can be used for filling one or more fluids to the first patterned microfluidic channel during the first pumping mechanism.

2. The microfluidic device as set forth in claim 1, wherein the first patterned microfluidic channel has a corrugated shape or a bell shape extending outwardly from a channel base.

3. The microfluidic device as set forth in claim 1, wherein the first patterned microfluidic channel has a membrane thickness in a range of 1 micrometer to 500 micrometers and a bump height larger than 1 micrometer.

4. The microfluidic device as set forth in claim 1, wherein the channel has an aspect ratio for a height to a width (h_{base}:w)) of less than 1.

5. The microfluidic device as set forth in claim 1, wherein the one or more fluids are air, liquid, drugs or reagents.

6. The microfluidic device as set forth in claim 1, wherein the first inlet can be sealed for a first reversible liquid displacement as opposed to the first pumping mechanism.

7. The microfluidic device as set forth in claim 1, further comprising:
(a) a second reservoir having a second patterned microfluidic channel; and
(b) a second inlet at one end of the second patterned microfluidic channel; and
(c) the channel connecting to another end of the second patterned microfluidic channel,
where strain applied to the second patterned microfluidic channel causes a second pumping mechanism of the one or more liquids flow from the second patterned microfluidic channel to the outlet via the channel, and
wherein the first and the second inlet can be used for filling the one or more liquids to the second patterned microfluidic channel during the second pumping mechanism.

8. The microfluidic device as set forth in claim 7, wherein the second can be sealed for a second reversible liquid displacement as opposed to the second pumping mechanism.

9. The microfluidic device as set forth in claim 7, wherein the second patterned microfluidic channel has a corrugated shape or a bell shape extending outwardly from a channel base.

10. The microfluidic device as set forth in claim 7, wherein the first and the second inlet are closed or open inlets.

11. The microfluidic device as set forth in claim 7, wherein the second patterned microfluidic channel has a membrane thickness in a range of 1 micrometer to 500 micrometers and a bump height larger than 1 micrometer.
